(19) United States
(12) Patent Application Publication
(10) Pub. No.: US 2024/0074830 A1
(43) Pub. Date: Mar. 7, 2024

(54) SUBJECT POSITIONING

(71) Applicant: UCL BUSINESS LTD, London Greater London (GB)

(72) Inventors: Bradley TREEBY, London Greater London (GB); Olivia WRIGHT, London Greater London (GB)

(21) Appl. No.: 18/261,411

(22) PCT Filed: Jan. 14, 2022
(86) PCT No.: PCT/IB2022/050292

§ 371 Date: Jul. 13, 2023

(30) Foreign Application Priority Data
Jan. 14, 2021 (GB) 2100449.4

(51) Int. Cl. A61B 90/18; A61B 5/00; A61N 7/00

(52) U.S. Cl. CPC A61B 90/18; A61B 5/702; A61N 7/00; A61N 2007/0026

(57) ABSTRACT

A subject positioning apparatus (10) is disclosed comprising a first part (200) and a second part (100). The first part (200) is configured to contact and conform with a first region of the subject's head and/or neck and to engage with and couple to the second part (100). The first and second part (200, 100) together hold the subject in a fixed position relative to the apparatus (10). The first and second part (200, 100) each further comprising a portion of a seal flange, the seal flange (201, 101) of the first and second part (200, 100) together configured to clamp to further medical equipment so that a region of the subject's head above the seal flange (201, 101) is received in the further medical equipment.

### TECHNICAL FIELD

The present invention relates to a method for positioning a subject for a medical procedure, and to apparatus for positioning a subject for a medical procedure.

### BACKGROUND

It is often necessary to accurately locate or position a subject before a medical procedure is performed. Accurate positioning of a subject enables a reference frame of any treatment device to be accurately related to a reference frame of the subject.

Examples of medical procedures where subject positioning is important are radiation oncology and high intensity focused ultrasound (HIFU) brain surgery. Any surgical procedure on the brain requires very careful planning, which may require accurate subject positioning.

In addition to surgical procedures, non-invasive stimulation of the brain may also require precise positioning of the subject's head, relative to apparatus performing the stimulation. Examples of non-invasive brain stimulation techniques include magnetic brain stimulation and ultrasound stimulation.

One method for positioning a subject's head before (and during) a medical procedure is to use a stereotactic frame. A stereotactic frame is attached to the subject's bone structure with fasteners (e.g. screws) and the frame can be coupled to a scanner or surgical apparatus coupled to the frame. Stereotactic frames are inherently invasive, in that they require secure attachment to the subject's cranium.

US2014/330417 discloses a medical immobilisation device for anchoring a subject during a CT or MRI scan. The device may be customised to the specific patient, e.g. by 3D printing at least some parts.

U.S. Pat. No. 7,283,861 discloses an example of a system for stimulating the brain with focused ultrasound.

An improved apparatus and method for positioning a subject during scanning and other medical procedures is desirable. More particularly, an apparatus and method for positioning a subject during ultrasonic brain stimulation and other transcranial ultrasound procedures is desirable.

### SUMMARY

According to a first aspect, there is provided a subject positioning apparatus, comprising:
a first part and a second part;
wherein the first part is configured to contact and conform with a first region of the subject's head and/or neck and to engage with and couple to the second part;
the second part is configured to contact and conform with a second region of the subject's head and/or neck; and
the first and second part together hold the subject in a fixed position relative to the apparatus.

The first part may be a neck part configured to contact and conform with a region of the subject's neck and/or a posterior region of the subject's head. The second part may be a face part configured to contact and conform with a region of the subject's face. In other embodiments the first part and second part may be configured to be placed on medial and lateral sides of the subject's head (rather than on anterior and posterior sides, as for face and neck parts).

The first part and the second part may be substantially rigid.

The first and second part may together be configured to fix the apparatus with a positional accuracy, relative to the subject's neurocranium, of less than 5 mm, less than 2 mm or less than 1 mm (i.e. the apparatus may be moveable by no more than 5 mm, 2 mm or 1 mm in any direction, relative to the subject's neurocranium). The first part and/or second part may conform to at least part of the subject's face with an accuracy of less than 2 mm (or less than 1 mm). The first part and/or second part may conform to a posterior region of the subject's head and/or neck with an accuracy of less than 2 mm (or less than 1 mm).

The first part may comprise a mating surface that is configured to engage with a corresponding mating surface of the second part. The first part and second part may each comprise a mating flange comprising the mating surface.

The mating flange and/or mating surfaces of the first part and second part may be substantially parallel to a frontal or sagittal plane of the subject (when the apparatus is in place on the subject's head). The mating flange of the first part and/or second part may comprise at least one shoulder feature for locating the first part with the second part in the frontal or sagittal plane (i.e. preventing relative movement between the first part and second part parallel to the plane of the mating flange).

The mating flange of the first part and second part may comprise at least one slot for receiving a fastener that is arranged to clamp the mating surfaces of the first part and second part together. The fastener may comprise a bolt and nut (e.g. a wingnut). The apparatus can be configured such that each fastener can slide laterally out of its respective slot (e.g. without removing the nut from the bolt), so that the first part can quickly be unclamped from the second part.

The first part and/or second part may be configured to engage with a zygomatic bone (cheek bone) of the subject. The first part and/or second part may be configured to engage with a squamous part of a frontal bone (forehead) of the subject. The first part and/or second part may be configured to engage with a nasal bone of the subject. The first part and/or second part may be configured to engage with a posterior region of the head (e.g. occipital bone) of the subject and/or with the subject's neck.

The apparatus may be located relative to the subject in a fixed position in the superior and inferior directions (of the subject) by engagement of the first part and/or second part with the nasal bone (which slopes in a first direction relative to the frontal plane), and by engagement of the first part and/or second part with the occipital bone (which slopes in a second direction relative to the frontal plane, the second direction opposite to the first direction). The apparatus may be located relative to the subject in a fixed position in the superior and inferior directions by engagement of the first part and/or second part with the nasofrontal angle.

The apparatus may be located relative to the subject in a fixed position in the medial and lateral direction by engagement of the face part and/or neck part with at least one of: the frontal bone; the sphenoid bones; and the temporal bones of the subject.

The apparatus may be located relative to the subject in a fixed position in the posterior and anterior directions by engagement of the first part and/or second part with the frontal bone, and engagement of the first part and/or second part with the occipital bone and/or the neck.

The first part and/or second part may comprise at least one eye hole for a subject to see through while the apparatus is in place on the subject's head. The first part and/or second part may comprise an open region exposing the subject's nostrils and mouth while the apparatus is in place on the subject's head (with the first part coupled to the second part about the subject's head). The first part and/or the second part may together comprise openings for the subject's ears.

The first part and second part may be customised to fit a specific subject. For example, the first part and second part may be configured to precisely engage with the subject's anatomy. The geometry of the subject's anatomy may be obtained by scanning of the subject (e.g. by MRI scanning, using optical camera images, or by x-ray tomography) (e.g. via surface mesh extraction from an MRI scan). Each of the first part and second part may be at least partly formed using a rapid prototyping method, such as by additive manufacturing or 3D printing. At least regions of the first part and second part that contact the subject, to locate the subject positioning apparatus, may be formed by additive manufacturing (e.g. 3D printing).

The apparatus may comprise a mirror (or more than one mirror), configured to allow the subject to see in an inferior or superior direction. A two mirror assembly may be used in some embodiments (for example, to look in an inferior direction). The mirror may be a removable mirror assembly that couples to the first and/or second part. The first and/or second part may comprise at least one rail for engaging with the mirror assembly along which the mirror assembly is moveable.

The apparatus may comprise a base portion, configured to couple to (or unitary with) at least one of the first part and second part. The base portion may comprise a base flange. The base flange may be configured to be received in a slot in further medical equipment to locate the apparatus (and the subject) relative to the further medical equipment. The further medical equipment may comprise a transcranial ultrasound device.

The transcranial ultrasound device may comprise a substantially hemispherical cap (or semi-ellipsoidal cap) for receiving the subject's head. The transcranial ultrasound cap may comprise an array of ultrasonic transducers configured to stimulate the subject's brain using focused ultrasound, for example. The transcranial ultrasound device and subject positioning assembly may be configured for use inside a magnetic resonance imaging scanner.

The first part and the second part may each comprise a seal flange. The seal flange of the first part and second part may together be configured to engage with a sealing assembly which encircles the subject's head. The sealing assembly may be substantially annular. The seal flange may be inclined at an angle to a transverse plane of the subject. The angle of inclination of the seal flange to the transverse plane may be between 10 and 30 degrees (or between 15 and 25 degrees, e.g. 20 degrees).

The seal flange may comprise one or more slots, each slot configured to receive a fastener for clamping the seal flange to further medical equipment (e.g. a transcranial ultrasound cap).

The subject positioning apparatus may be configured for use in an MRI scanner.

According to a second aspect, there is provided a subject positioning apparatus, comprising:
a first part and a second part;
wherein the first part is configured to contact and conform with a region of the subject's head and/or neck, and to engage with and couple to a second part;
the first part is configured to hold the subject, when coupled to the second part, in a fixed position relative to the apparatus;
the first and second part each further comprising a portion of a seal flange, the seal flange of the first and second part together configured to clamp to further medical equipment so that a region of the subject's head above the seal flange is received in the further medical equipment.

The further medical equipment may comprise a cap with at least one ultrasonic transducer (such as a transcranial ultrasound cap).

The second part may be configured to contact and conform with a region of the subject's head and/or neck.

The first part may be a neck part configured to contact and conform with a region of the subject's neck and/or a posterior region of the subject's head. The second part may be a face part configured to contact and conform with a region of the subject's face. In other embodiments the first part and second part may be configured to be placed on medial and lateral sides of the subject's head (rather than on anterior and posterior sides, as for face and neck parts).

The first part and the second part may be substantially rigid.

The first and second part may together be configured to fix the apparatus with a positional accuracy, relative to the subject's neurocranium, of less than 5 mm, less than 2 mm or less than 1 mm (i.e. the apparatus may be moveable by no more than 5 mm, 2 mm or 1 mm in any direction, relative to the subject's neurocranium). The first part and/or second part may conform to at least part of the subject's face with an accuracy of less than 2 mm (or less than 1 mm). The first part and/or second part may conform to a posterior region of the subject's head and/or neck with an accuracy of less than 2 mm (or less than 1 mm).

The first part may comprise a mating surface that is configured to engage with a corresponding mating surface of the second part. The first part and second part may each comprise a mating flange comprising the mating surface.

The mating flange and/or mating surfaces of the first part and second part may be substantially parallel to a frontal or sagittal plane of the subject (when the apparatus is in place on the subject's head). The mating flange of the second part and/or face part may comprise at least one shoulder feature for locating the first part with the second part in the frontal or sagittal plane (i.e. preventing relative movement between the first part and second part parallel to the plane of the mating flange).

The mating flange of the first part and second part may comprise at least one slot for receiving a fastener that is arranged to clamp the mating surfaces of the first part and second part together. The fastener may comprise a bolt and nut (e.g. a wingnut). The apparatus can be configured such that each fastener can slide laterally out of its respective slot (e.g. without removing the nut from the bolt), so that the first part can quickly be unclamped from the second part.

The first part and/or second part may be configured to engage with a zygomatic bone (cheek bone) of the subject. The first part and/or second part may be configured to engage with a squamous part of a frontal bone (forehead) of the subject. The first part and/or second part may be configured to engage with a nasal bone of the subject. The first part and/or second part may be configured to engage with a posterior region of the head (e.g. occipital bone) of the subject and/or with the subject's neck.

The apparatus may be located relative to the subject in a fixed position in the superior and inferior directions (of the subject) by engagement of the first part and/or second part with the nasal bone (which slopes in a first direction relative to the frontal plane), and by engagement of the first part and/or second part with the occipital bone (which slopes in a second direction relative to the frontal plane, the second direction opposite to the first direction). The apparatus may be located relative to the subject in a fixed position in the superior and inferior directions by engagement of the first part and/or second part with the nasofrontal angle.

The apparatus may be located relative to the subject in a fixed position in the medial and lateral direction by engagement of the first part and/or second part with at least one of: the frontal bone; the sphenoid bones; and the temporal bones of the subject.

The apparatus may be located relative to the subject in a fixed position in the posterior and anterior directions by engagement of the first part and/or second part with the frontal bone, and engagement of the first part and/or second part with the occipital bone and/or the neck.

The first part and/or second part may comprise at least one eye hole for a subject to see through while the apparatus is in place on the subject's head. The first part and/or second part may comprise an open region exposing the subject's nostrils and mouth while the apparatus is in place on the subject's head (with the first part coupled to the second part about the subject's head). The first part and/or the second part may together comprise openings for the subject's ears.

The first part and second part may be customised to fit a specific subject. For example, the first part and second part may be configured to precisely engage with the subject's anatomy. The geometry of the subject's anatomy may be obtained by scanning of the subject (e.g. by MRI scanning, using optical camera images, or by x-ray tomography) (e.g. via surface mesh extraction from an MRI scan). Each of the first part and second part may be at least partly formed using a rapid prototyping method, such as by additive manufacturing or 3D printing. At least regions of the first part and second part that contact the subject, to locate the subject positioning apparatus, may be formed by additive manufacturing (e.g. 3D printing).

The apparatus may comprise a mirror (or more than one mirror), configured to allow the subject to see in an inferior or superior direction. A two mirror assembly may be used in some embodiments (for example, to look in an inferior direction). The mirror may be a removable mirror assembly that couples to the first and/or second part. The first and/or second part may comprise at least one rail for engaging with the mirror assembly along which the mirror assembly is moveable.

The apparatus may comprise a base portion, configured to couple to (or unitary with) at least one of the first part and second part. The base portion may comprise a base flange. The base flange may be configured to be received in a slot in further medical equipment to locate the apparatus (and the subject) relative to the further medical equipment. The further medical equipment may comprise a transcranial ultrasound device.

The transcranial ultrasound device may comprise a substantially hemispherical cap (or semi-ellipsoidal cap) for receiving the subject's head. The transcranial ultrasound cap may comprise an array of ultrasonic transducers configured to stimulate the subject's brain using focused ultrasound, for example. The transcranial ultrasound device and subject positioning assembly may be configured for use inside a magnetic resonance imaging scanner.

The seal flange may be configured to engage with a sealing assembly which encircles the subject's head. The sealing assembly may be substantially annular. The seal flange may be inclined at an angle to a transverse plane of the subject. The angle of inclination of the seal flange to the transverse plane may be between 10 and 30 degrees (or between 15 and 25 degrees, e.g. 20 degrees).

The seal flange may comprise one or more slots, each slot configured to receive a fastener for clamping the seal flange to further medical equipment (e.g. a transcranial ultrasound cap).

The subject positioning apparatus may be configured for use in an MRI scanner.

In either the first or second aspect, the first part may comprise a rigid portion that comprises or consists of a rigid material and a contact part that comprises a softer material. The contact part may be in direct contact with the subject. The second part may comprise a rigid portion that comprises or consists of a rigid material and a contact part that comprises a soft material. The contact part (of the first and/or second part) may be formed from a cast or impression of the shape of the subject.

Features of the second aspect (including optional features) may also be combined with the first aspect. Features of the first aspect (including optional features) may also be combined with the second aspect.

According to a third aspect of the invention, there is provided a sealing assembly for sealing engagement with a transcranial ultrasound cap and a subject's head, for retaining an ultrasonic transmission medium in contact with the subject's head and in contact with transducers of the transcranial ultrasound cap.

The sealing assembly may be customised to fit a specific subject. The sealing assembly may be at least partly formed using an additive manufacturing process (such as 3D printing). For example, an inner seal of the sealing assembly may be configured to precisely engage with the subject's anatomy after the subject has been scanned (e.g. by MRI scanning, using optical camera images, or by x-ray tomography) to determine the geometry of their anatomy (e.g. via surface mesh extraction from an MRI scan). A portion of the sealing assembly (e.g. the inner seal) may be formed from a cast or impression of the shape of the subject. The cast or impression need not be formed directly from the subject. In some embodiments a scan may be used to determine the geometry of the subject, and a cast made that reflects this geometry (e.g. using rapid prototyping techniques).

The inner seal may be positioned precisely relative to the subject by the subject positioning apparatus (of the first or second aspects), and the inner seal can consequently be precisely formed to conform to the contours of the subject at that position. The inner seal may be configured to seal with (or near, such as within 10 mm or within 20 mm) a planar cross section of the skin surface surrounding the neurocranium with maximum cross sectional area. Such a selection may contribute towards the sealing assembly achieving a good seal.

The sealing assembly may be configured to be clamped between a subject positioning apparatus (of the first or second aspects) and the transcranial ultrasound cap in order to form a seal with the cap and to be retained in sealing engagement with the subject.

The sealing assembly may comprise an outer sealing ring and an inner seal. The outer sealing ring may be configured to seal with a transcranial ultrasound cap, and the inner seal may be configured to seal against the subject (e.g. their head).

The outer sealing ring may comprise a substantially flat annular element. The outer sealing ring may be configured to be clamped between the subject positioning apparatus and the transcranial ultrasound cap in order to form a seal with the transcranial ultrasound cap. An additional further sealing element (e.g. a compliant annular sealing element) may be provided between the outer sealing ring and the transcranial ultrasound cap.

The sealing assembly may be configured for use in an MRI scanner.

The outer sealing ring may be configured to retain an inner seal in sealing engagement with the subject's head. The outer sealing ring may be substantially rigid and may be formed from a polymeric material (e.g. with a Shore D hardness greater than 60).

The inner seal may comprise a compliant portion and a retaining portion. The compliant portion may be configured to contact the subject's head and to form a seal therewith. The retaining portion may be configured to retain the compliant portion in sealing engagement with the subject's head. Both the compliant portion and the retaining portion of the inner sealing ring may be configured to encircle the subject's head.

The inner seal may have a radial extent of less than 25 mm, or less than 20 mm, or less than 15 mm. The sealing assembly may have an essentially flat annular shape, with a thickness of 20 mm or less (or 10 mm or less).

The compliant portion may consist of or comprise a material with a Shore A hardness of less than 50, or less than 40, or less than 30. The retaining portion may consist of or comprise a material with a Shore D hardness of greater than 60. The compliant portion may be attached to the retaining portion with adhesive. The inner seal may be formed using a two material additive manufacturing process so that the compliant portion is manufactured with the retaining portion, but using a different material.

The outer sealing ring may be configured to form a seal with the further medical equipment (e.g. a transcranial ultrasound cap), for example when clamped between the further medical equipment and the subject positioning apparatus. The outer sealing ring may be configured for sealing engagement with the inner seal.

According to an aspect, there is provided a positioning cap for use with the apparatus of the first or second aspect. The positioning cap is configured to temporarily engage with the first part or the second part, so as to better position the subject for the first and second parts to be coupled together. The positioning cap may be configured to conform with the subject's upper cranium, and may be subject specific. The positioning cap may comprise a head part that has two halves. The head part may be received in an outer ring that retains two halves of the head part together. The outer ring may be configured to be coupled to the seal flange (of the first part and/or second part).

According to a fourth aspect, there is provided a system comprising the subject positioning assembly and the sealing assembly, including any of the optional features described herein with reference to the other aspects.

According to a fifth aspect, there is provided a method of locating a subject relative to medical equipment, using an apparatus according to the first or second aspect (including any optional features thereof). The method comprises:
placing the first part in conformal contact with a portion of the subject's head and/or neck;
placing the second part in contact with a portion of the subject's head and/or neck; fixing the second part to the first part.

According to a sixth aspect, there is provided a method of locating a subject relative to medical equipment, comprising:
placing a first part in conformal contact with a portion of the subject's head and/or neck;
placing a second part in conformal contact with a portion of the subject's head and/or neck;
fixing the second part to the first part.

The method according to the sixth aspect may comprise using apparatus according to the first or second aspect, including any of the optional features thereof.

The following optional features are applicable to either the fifth aspect or the sixth aspect.

The first part may be a neck part, configured for conformal contact with a posterior portion of the subject's head and/or neck. The second part may be a face part, configured for conformal contact with a portion of the subject's face.

The medical equipment may comprise a brain stimulation device or a device configured to perform a surgical or therapeutic procedure on the subject's brain (e.g. a radiation oncology device, a high intensity focused ultrasound device, a surgical robot, a transcranial ultrasound cap, etc)

The method may exclude performing any treatment or surgical steps on the subject, and relate only to securely and accurately positioning the subject ready for treatment.

The method may comprise subsequently fixing the first/neck part to a base part. The base part may be according to the first aspect or second aspect, including any of the optional features thereof.

The method may comprise slipping a seal assembly onto the subject's head adjacent to the second/face part and first/neck part. The seal assembly may be according to the third aspect, including any of the optional features thereof.

The method may comprise clamping the first/neck part and second/face part (subject positioning assembly) to the medical equipment, with the seal assembly clamped between the subject positioning assembly and the medical equipment.

Prior to fixing the first part to the second part, a positioning cap may be coupled to the first part or second part. The positioning cap may conform with a top portion of the subject's cranium.

After fixing the first part to the second part, the positioning cap may be detached from the first part or second part.

According to a seventh aspect, there is provided a method of manufacturing a subject positioning apparatus according to the first or second aspect, a sealing assembly according to the third aspect and/or a system according to the fourth aspect, comprising:
i) scanning the subject to determine the geometry of their head and/or neck;
ii) determining a matching shape for regions of the apparatus that are configured to conform with the subject;
iii) manufacturing the apparatus in dependence on the results of step ii).

The scanning of the subject may be by MRI, optical scanning and/or CT x-ray scanning. The manufacturing of the apparatus may comprise using additive manufacturing (e.g. 3D printing).

The features of each aspect (including optional features) may be combined with those of any other aspect.

### BRIEF DESCRIPTION OF THE DRAWINGS

Example embodiments will be described, by way of example only, with reference to the drawings, in which:

FIG. 1 shows a schematic view of a subject head and transcranial ultrasound cap in the sagittal plane and frontal plane;

FIG. 2 shows a sectional view of a transcranial ultrasound cap, sealing assembly and subject head in the frontal plane;

FIG. 3 shows an example embodiment comprising a face part and neck part;

FIG. 4 shows detail views of the embodiment of FIG. 3, illustrating how the face part and neck part engage each other;

FIG. 5 shows the example neck part and a base part according to an embodiment;

FIG. 6 shows a sealing assembly in accordance with an embodiment;

FIG. 7 shows an example transcranial ultrasound cap according to an embodiment;

FIG. 8 shows an exploded diagram of the elements shown in FIGS. 3 to 7;

FIG. 9 shows the elements of FIG. 8 in a partially assembled state, with the face and neck part coupled together, and the neck part coupled to the base part;

FIG. 10 shows an apparatus in a fully assembled state, with the seal assembly clamped between the transcranial ultrasound cap and the neck part and face part;

FIG. 11 shows a flow chart of a method according to an embodiment;

FIG. 12 shows an exploded view of an embodiment comprising a positioning cap which is used in intermediate positioning of the subject to aid location of the subject in the face and neck part;

FIG. 13 shows a side view of an assembled base part, neck part and face part, with a positioning cap spaced apart from the assembled base part, neck part and face part; and

FIG. 14 shows an exploded view of an embodiment comprising cast rubber parts for improving comfort.

### DETAILED DESCRIPTION

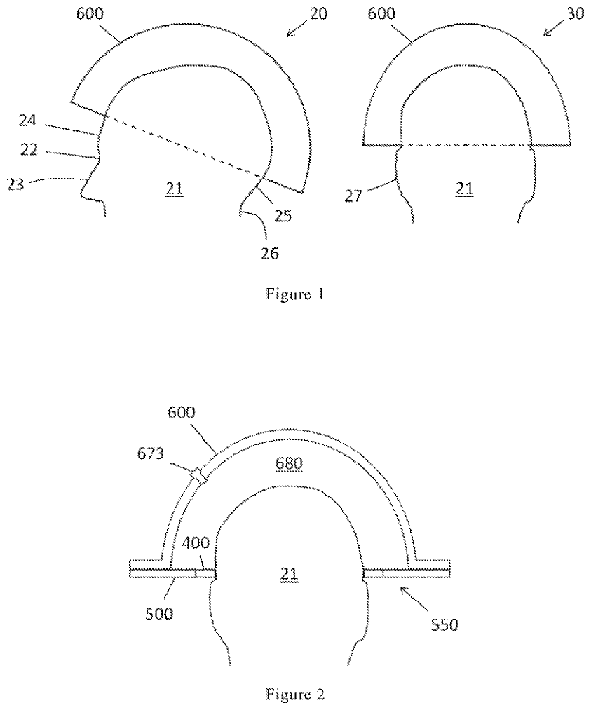

Referring to FIG. 1, a subject 21 is shown in the sagittal plane 20 and frontal plane 30. A transcranial ultrasound cap 600 is shown, at least partially surrounding the subject's neurocranium, so that focused ultrasound can be directed into the subject's brain. The cap 600 may be substantially hemispherical or substantially hemi-ellipsoidal. The cap 600 comprises an array of transducers coupled to an inner surface of the cap, which is spaced from the subject's head (e.g. by 30-50 mm, in order to accommodate variation in subject anatomy).

As shown in FIG. 2, in order to couple ultrasonic energy from an ultrasound transducer 673 to the subject's head 21, it is necessary to fill the space 680 between the transducer 673 and the subject's head with a suitable ultrasonic transmission medium that is sufficiently well matched in ultrasonic transmission properties to the subject. Water is a convenient and suitable ultrasonic transmission medium.

In order to maintain the ultrasonic transmission medium between the transducers and subject, a liquid (e.g. water) tight seal must be provided between the transcranial ultrasound cap 600 and the subject 21. Such a seal may be provided by a seal assembly 550 that is coupled to the transcranial ultrasound cap 600. The seal assembly 550 may comprise an inner seal 400 in contact with the subject and an outer seal 500 that retains the inner seal 400 and is configured for sealing engagement with the transcranial ultrasound cap 600. A further seal element (not shown) may be provided to ensure there is a seal between the seal assembly 550 and the transcranial ultrasound cap 600.

In order to ensure that ultrasonic energy from the transcranial ultrasound cap 600 is directed to the appropriate location in the subject's brain, it is of critical importance that the subject be accurately located, relative to the transcranial ultrasound cap 600 (as briefly discussed in the background).

FIG. 1 shows some features of the subject's anatomy that may be used by a subject positioning apparatus to rigidly locate the transcranial ultrasound cap 600 in a specific position relative to the subject 21. For example, a subject positioning apparatus may engage the subject's nasofrontal angle 22, between the subject's nose 23 and forehead 24, in order to prevent movement of the subject positioning apparatus in the superior and inferior directions. A subject positioning apparatus may also engage with a posterior portion of the subject's head 25 (e.g. the occipital bone) or their neck.

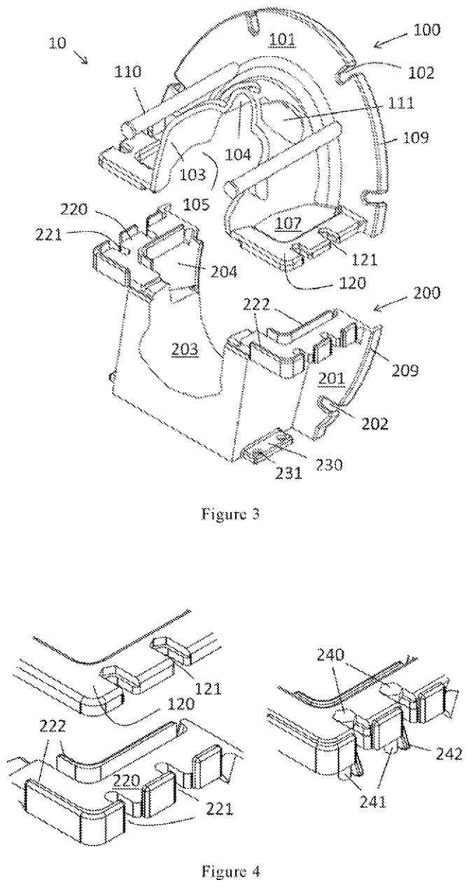

FIG. 3 shows an example subject positioning apparatus 10, comprising a first/neck part 200 and second/face part 100. The face part 100 and neck part 200 are separable parts that are configured to be coupled about the subject's head, with the face part 100 on the anterior of the head and the neck part 200 on the posterior of the head/neck.

Each of the face part 100 and the neck part 200 may comprise a unitary part, which may be formed by an additive manufacturing process such as 3D printing. In some embodiments the face part 100 and/or neck part 200 may comprise portions that are manufactured using 3D printing (that are tailored to the specific anatomy of the subject) and portions that are not subject specific (which may economically be manufactured with other manufacturing processes).

The face part 100 comprises regions 104, 103 configured to conform to the geometry of the subject's face and head, and engages with the subject's nose, forehead and cheekbones, as well as the sides of the subject's head. Regions 104, 103 may conform to the subject with an accuracy of 2 mm or less (or 1 mm or less). The face part 100 comprises eye holes 111 for the subject to see out of, and a cut out region that exposes the subject's mouth and nostrils so that they can breathe freely and talk while the face part 100 is in place (e.g. to provide information about any effects of brain stimulation). Further cut-out regions 105 are provided to comfortably accommodate the subject's ears.

The neck part 200 comprises a region 203 that is configured to conform precisely (e.g. with an accuracy of 2 mm or less or 1 mm or less) to the geometry of the subject's neck and/or occipital bone. The neck part 200 and the face part 100 are therefore configured to clamp together on either side of the subject's head to securely (but comfortably) locate the apparatus 10 relative to the subject. The accuracy with which the face part 100 and neck part 200 conform to the subject's head, in combination with the differently oriented surfaces of the subject's head (and the underlying bone structure) result in the apparatus 10 being fixed in place with an accuracy of less than 5 mm, preferably less than 2 mm, or less than 1 mm.

The face part 100 comprises a mating flange 120 (which, in this embodiment, is configured to be substantially parallel to a frontal plane of the subject) with a mating surface configured to engage with a corresponding mating surface of a mating flange 220 of the neck part 200. The mating flange 120 of the face part 100 comprises slots 121 for receiving fasteners for clamping the mating flange 120 of the face part 100 to the mating flange 220 of the neck part 200. Corresponding slots 221 are provided in the mating flange 220 of the neck part 200. The mating flange 220 of the neck part 200 comprises a shoulder 222 for locating the face part 100 relative to the neck part 200 in the frontal plane when the face part 100 and neck part 200 are engaged. In other embodiments the mating flange may be differently oriented.

FIG. 4 shows the engagement of the mating flanges 120, 220 in more detail, and shows the flanges 120, 220 in a mated configuration with fasteners 240 clamping the face part 100 to the neck part 200. The fasteners comprise bolts 241 with wing nuts 242. The hexagonal heads of the bolts 241 are captured by the slots 121 in the flange 120, so that the bolts are prevented from rotating. The bolts can be slid away from engagement with the face part 100 and neck part 200 via the slots 121, 221 without removing the wing nuts 242 from the bolts 241. This allows for relatively quick disassembly of the face part 100 from the neck part 200. It will be appreciated that other forms of removable fastener can be used to couple the face part 100 to the neck part 200, such as lever operated cams, and toggle clamps or similar locking linkage arrangements.

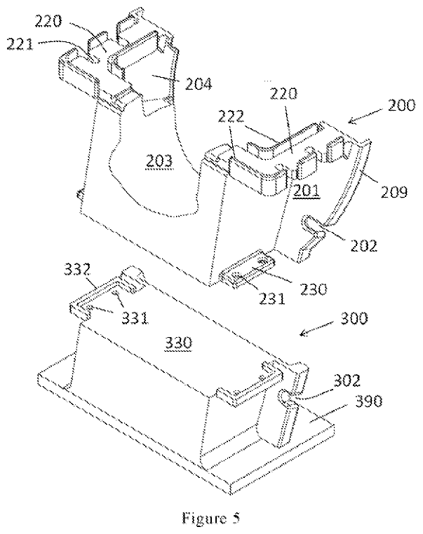

FIG. 5 shows the neck part 200 with a base part 300, which is used to couple the neck part 200 to the transcranial ultrasound cap 600. In some embodiments the base part 300 may be unitary with the neck part 200 (and may consequently be referred to as a base portion). The neck part 200 in the example embodiment comprises lower flanges 230 on opposing sides, which are received in a shoulder 332 of a corresponding flange of the base part 300. Holes 231, 331 are provided in the lower flange 230 and corresponding flange of the base part for fastening the neck part 200 to the base part 300 (e.g. with screws received in threads in the base part 300). The base part 300 may comprise at least one slot 302 for receiving a fastener that clamps the base part 300 to the transcranial ultrasound cap 600. The base part 300 further comprises a base flange 390 that extends in the frontal plane in the superior direction, and which is configured to be received in a corresponding slot of the transcranial ultrasound cap 600 to align the subject positioning assembly slots 102, 202, 302 with corresponding slots of the transcranial ultrasound cap 600 (ready for clamping together with fasteners in the slots 102, 202, 302, 602).

The face part 100 and neck part 200 both comprise a seal flange 101, 201 for engagement with a seal assembly. The seal flange 101, 201 may be inclined, so that rather than being substantially parallel to the transverse plane of the subject (i.e. perpendicular to the mating flanges 120, 220), the seal flange is at an angle. The angle of inclination may be between 10 and 30 or between 15 and 25 degrees from the transverse plane of the subject (e.g. 20 degrees).

The inclination angle of the seal flange 101, 201 may correspond with an angled transcranial ultrasound cap 600, which may have advantages. The air-filled frontal sinus blocks ultrasound signals from coupling to the brain from a significant region at the front of the head, so tilting a substantially hemispherical array of transducers backward will improve coverage. There are more substantial variations between subjects below the external occipital protuberance, which do not fit well to an ellipse, limiting the degree to which this tilt is useful. The inventors have identified that an angle of between 15 and 25 degrees strikes a good compromise between these competing factors.

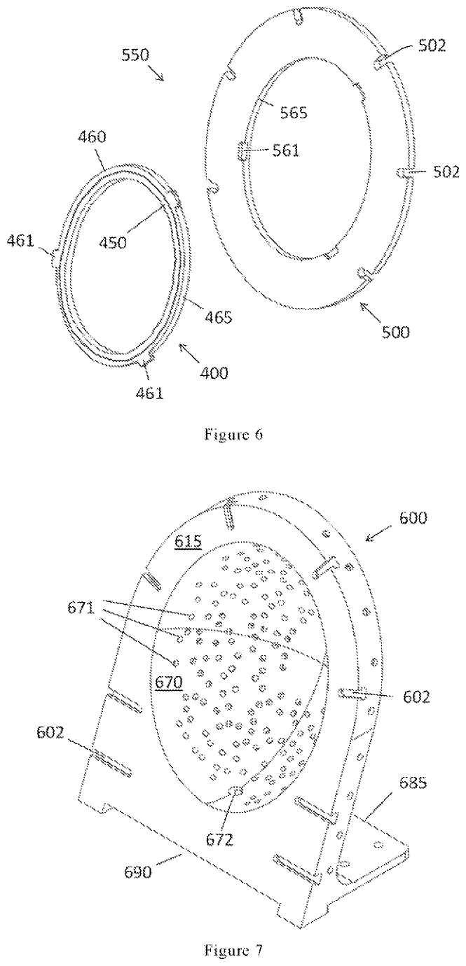

The seal flange of the neck part and face part are together configured to engage a substantially flat annular seal assembly (e.g. as shown in FIG. 6) that is configured to encircle the subject's head. The seal flange 101, 201 comprises a flat annular surface that is configured to clamp the seal assembly to the further medical equipment (e.g. transcranial ultrasound cap). The seal flange 101, 201 further comprises a shoulder 109, 209 for retaining the seal assembly and constraining movement parallel to a plane of the seal flange 101, 201 (this feature may also/alternatively be provided on the transcranial ultrasound cap). The seal flange 101, 201 is provided with slots 102, 202 for receiving fasteners that may be used to clamp the seal flange 101, 201 to the transcranial ultrasound device.

FIG. 6 shows a sealing assembly 550, comprising an inner seal 400 and outer sealing ring 500.

The outer sealing ring 500 may comprise a substantially flat annulus of a hard polymer material (e.g. with a Shore D hardness greater than 60). The outer sealing ring 500 comprises slots 502 corresponding with the slots 102, 202 of the sealing flange 101, 201. The outer sealing ring 500 comprises a surface 565 that engages with the inner seal 400 to locate it radially and recesses 561 that locate the inner seal circumferentially (by receiving corresponding protrusions 461 of the inner seal 400). The surface 565 may be cylindrical or elliptical (or may be tailored to the subject's head shape). There are three unequally spaced recesses 561 in this example so that the inner seal 400 can only be received in the correct orientation in the outer seal 500 (but this is not essential, and in some embodiments the recesses may be equally spaced).

The inner seal 400 is configured to conform to the subject's head, so is roughly annular, but may be closer in conformation to an elliptical annulus than a circular annulus. The inner seal 400 comprises an inner sealing ring (or compliant portion) 450 that consists of a soft material (e.g. with a Shore A hardness of less than 50, or less than 40, or less than 30). The inner seal 400 is configured to match the geometry of the subject's head so that it forms a comfortable and reliable seal, while at the same time taking a minimum of contact space on the subject's head. This is significant, because a larger seal blocks access to more of the subject's head, potentially impacting the performance of a transcranial ultrasound device coupled to the seal assembly 550. The inner seal 400 further comprises a retaining ring 460, having an outer surface 465 for engaging with the corresponding surface 565 of the outer sealing ring 500. The retaining ring 460 comprises a harder polymer material than the inner sealing ring 450 (and may have a Shore D hardness greater than 60).

Making the sealing assembly in two parts, with an outer sealing ring 500 and an inner seal 400 may reduce the amount of the sealing assembly that needs to be customised for a specific subject (i.e. potentially just the inner seal).

FIG. 7 shows a further medical equipment in the form of a transcranial ultrasound cap 600. The transcranial ultrasound cap 600 comprises a hemi-ellipsoidal bowl 670 for receiving and supporting an array of ultrasonic transducers. The ultrasonic transducers are received in holes 671 in the bowl 670. The position of the ultrasonic transducers may be arranged for optimal performance by positioning them in quasi-random positions in specific regions of the bowl 670. The transcranial ultrasound cap 600 comprises a sealing surface 615 for engaging with the outer sealing ring 500 of the seal assembly 550. The cap 600 further comprises slots 602 for receiving fasteners that couple the cap 600 to the subject positioning apparatus 10 with the sealing assembly in-between. The slots 602 may be configured to receive hex bolt heads and prevent rotation of the bolt in the slot (so that wing nuts can be tightened against the seal flange 101, 201 without worrying about rotation of the bolt). The cap 600 comprises a lower flange 685 for securing the cap 600 (e.g. to a bed of an MRI scanner).

The transcranial ultrasound cap 600 may comprise at least one fluid port 672 for communicating water (or another suitable ultrasonic transmission medium) into the space 680 between the cap 600 and the subject's head 21.

FIG. 8 shows an exploded view of the parts shown in FIGS. 3 to 7, and also shows a mirror part 350. The mirror part 350 is configured to be received on rails 110 of the face part 100, the rails extending in an inferior direction from the seal flange 101. The mirror part 350 comprises a reflective element 351 that can be rotated to direct the subject's view in the superior direction (e.g. so that they can see out of an MRI scanner). A further mirror element may be provided (for example to fold an optical path to enable viewing in the inferior direction).

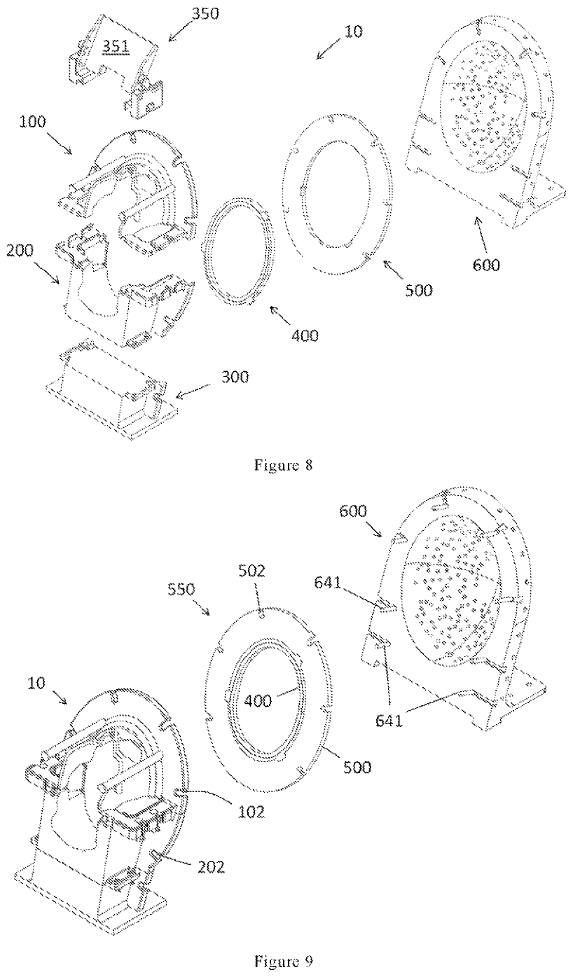

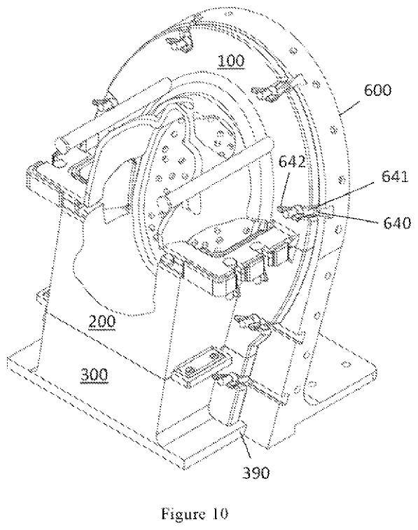

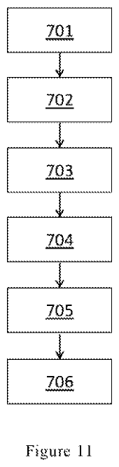

FIG. 9 shows the subject positioning apparatus 10 in an assembled condition and attached to the base part 300. Bolts 641 are received in the slots of the transcranial ultrasound cap 600, ready to be received in slots 102, 202, 302, 502. The seal assembly 550 is assembled, with the inner seal 400 received in the outer sealing ring 500. The bolts 641 may also be inserted after the components are fully assembled as shown in FIG. 10.

FIG. 10 shows the subject positioning apparatus 10 clamped to the transcranial ultrasound cap 600 by bolts 641 and wingnuts 642. The base flange 390 is received in the slot 690. The seal assembly 500 is clamped between the subject positioning apparatus 10 and the cap 600 (and would make a seal to a subject's head, in use).

FIG. 11 shows a flow diagram of a method of using the parts shown in FIGS. 3 to 10, comprising steps 701 to 706. In step 701 the neck part is fixed to base part 300 (e.g. by screws received in holes 231, 331). Step 701 may alternatively take place at any point prior to step 706. In step 702 the neck part 200 is placed in conformal contact with a posterior region of the subject's head and/or neck. In step 703 the face part 100 is placed in conformal contact with the subject's face. Alternatively, the order of step 702 and 703 may be reversed. At step 704 the face part 100 is fixed to the neck part 200 (e.g. using bolts in the slots 121, 221, as shown in FIG. 4). At step 705 the assembled seal assembly 550 is slipped onto the subject's head adjacent to the face and neck parts 100, 200. At step 706 the face and neck parts 100, 200 are clamped to the transcranial ultrasound cap 600, with the seal assembly 550 between. The subject is consequently accurately located with respect to the transcranial ultrasound cap 600, and the space 680 between the transcranial ultrasound cap 600 and subject 21 can be filled with couplant (e.g. water).

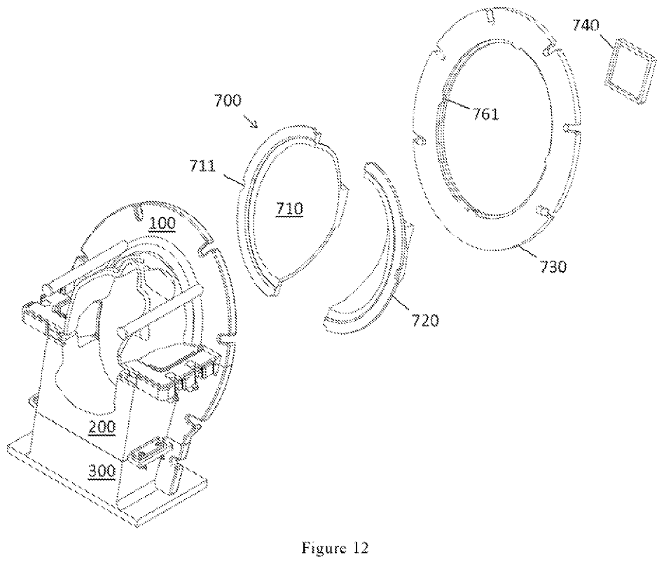

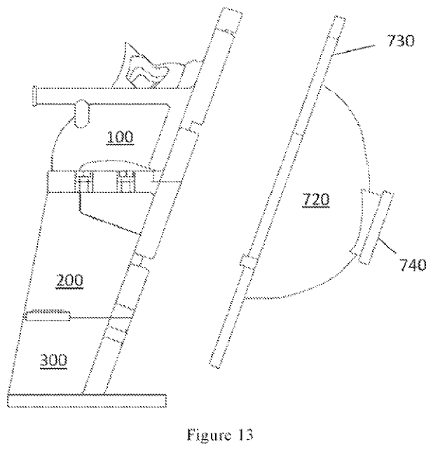

FIGS. 12 and 13 shows an embodiment comprising the face part 100, neck part 200 and base part 300 described with reference to FIGS. 2 to 10, and further comprising a positioning cap 700. The positioning cap 700 is used to help position the subject properly in the face part 100 or neck part 200 (depending on the order of assembly) before the face part 100 and neck part 200 are fixed together.

The positioning cap 700 comprises a head part 710, 720 that is split into two halves: a right half 710 and left half 720 (left and right here defined with reference to the subject). The head part 710, 720 is configured to conform with subject's upper cranium. The two halves 710, 720 may be formed using a rapid prototyping method and customised for the subject (as already described with reference to the face part and neck part). Splitting the head part into two halves 710, 720 enables it to be removed from the subject's head when they are positioned relatively close to the transcranial ultrasound cap 600.

The positioning cap 700 further comprises an annular outer ring 730 that need not be subject specific The outer ring 730 receives the head parts 710, 720 in a groove defined in an interior surface of the outer ring, and retains the head parts 710, 720 in engagement with each other. The head parts 710, 710 may comprise a location feature 711 that mechanically interacts with a corresponding locating feature 761 of the outer ring 730 to define a specific orientation of the head parts 710, 720 with the outer ring 730. In this example the location feature 711 on the head parts is a recess, receiving a protrusion 761 of the outer ring, but the opposite approach can be used (or any other mechanical locating approach).

The positioning cap 700 further comprises an outer cap 740, which acts to reinforce the engagement of the head parts 710, 720 by the outer ring 730. The outer cap 740 and head parts 710, 720 are provided with corresponding features for mechanical engagement therebetween. In this example a square protrusion is defined by features of both head parts 710, 720, and the outer cap 740 comprises a corresponding recess for receiving the square protrusion. Other features can be used for similar effect. For example, the protrusion need not be square—any shape will do. Alternatively, the outer cap 740 may be provided with dowels that are received in corresponding holes defined in the head parts 710, 720.

All of the parts 710, 720, 730, 740 of the positioning cap 700 may be substantially rigid, and may be formed from a polymeric material (e.g. with a Shore D hardness greater than 60).

The assembly order for an embodiment employing a positioning cap 700 may be modified from that described with reference to FIG. 11. In one method, the subject's head is first placed in the neck part 200 (or face part 100). The positioning cap 700 is subsequently placed on the subject's head. The positioning cap 700 is subsequently attached to the neck part 200 (or face part 100, if that is what is already placed on the subject). This can be achieved using fasteners that clamp the outer ring 730 to the seal flange 201 (or seal flange 101) via the slots 121. Once the positioning cap 700 is fixed to the neck part 200 (or face part 100) the subject will be properly aligned so that the face part 100 and neck part 200 can be fixed together on the subject. The positioning cap 700 can subsequently be removed. The face and neck parts 100, 200 can then be clamped to the transcranial ultrasound cap 600, with a seal assembly 550 between.

The positioning cap 700 is an optional part, which may be useful for some subjects. For some subjects it is relatively easy to get the face and neck parts 100, 200 assembled without the positioning cap 700. Some subjects may have a lot of hair, which will tend to make the positioning cap 700 less useful (because more offset between the subject's cranium and the cap 700 will be necessary to accommodate the hair, reducing any positional accuracy obtained by using the positioning cap 700).

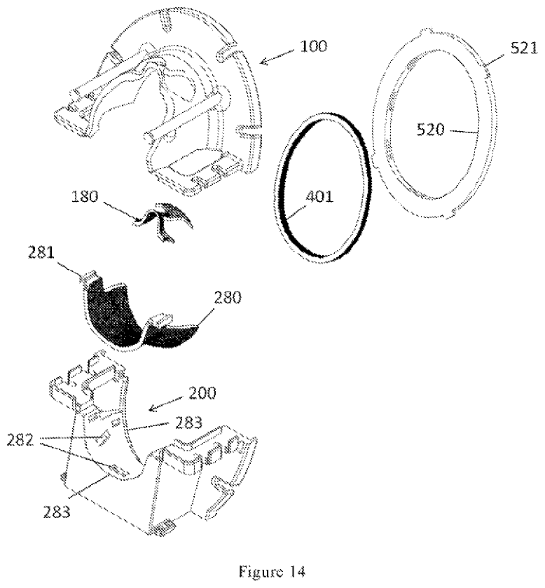

FIG. 14 shows an example embodiment in which portions of the face part 100 and neck part 200 comprise a soft polymeric material (e.g. with a shore A hardness of less than 50, less than 40, or less than 30). In this example the face part 100 and neck part 200 may be substantially as already described (made from harder polymeric material, for example by additive manufacture etc), but portions in contact with the subject are replaced by parts formed from softer material. The face part 100 in this example comprises contact part 180, and the neck part 200 comprises contact part 280. Using a softer material for these contact parts may significantly improve comfort for the user, without compromising on accuracy (which is at least partly conferred by the stiffness of the non-contacting parts).

The face contact part 180 may be configured to contact the nasal bone and occipital bone so as to engage with the nasofrontal angle. The face contact part 180 may comprise less than 75% of a subject facing surface area of the face part 100, or less than 50% of the subject facing surface area of the face part. A subject facing area may be defined as an inward facing surface that is immediately adjacent to the subject (and which typically has customised geometry to conform with the subject).

The neck contact part 280 may be configured to contact a posterior region of the subject's neck and/or a portion of the subject's neck. The neck contact part 280 may comprise more than 50% of a subject facing area of the neck part 200. The neck contact part 280 may improve comfort of the neck part 200, by helping prevent the neck part 200 from digging into the subject's neck. The neck contact part 280 may extend to encompass edges 283 of the neck part 200 that may dig into the subject's neck.

The neck contact part 280 may comprise a mechanical location feature 281 that mechanically interacts with a corresponding feature of a rigid portion of the face part 100 or neck part 200 to locate and/or engage the neck contact part therewith. In this example a flange 281 is provided on the neck contact part 280 that engages the rigid portion of the face part 100, but other arrangements are also possible. For example, protrusions may be provided on the back of the neck contact part 280 that are received in corresponding recesses on the rigid portion of the neck part 200.

Each of the contact parts 180, 280 may be formed by an impression or cast of the shape of the subject. In some embodiments, the contact parts 180, 280 may be formed directly by taking a cast of the subject (for example, using a subject specific former that is made using rapid prototyping). In other embodiments, an impression of the subject may be used as an intermediate form for casting at least one of the contact parts 180, 280. In other embodiments, a cast may be made from parts that have been manufactured to correspond with the geometry of the subject. In the example embodiment, the neck contact part 280 is formed by pouring a liquid rubber onto the rigid portion of the neck part 200, with a 3D printed former that mimics the shape of the subject. The neck contact part 280 is subsequently retained in place, at least in part, by protrusions of the neck contact part 280 received in recesses 282 in the rigid portion of the neck part 200. The face contact part 180 can be made with a similar method, and retained in place in a similar way.

The inner seal 401 depicted in FIG. 14 may similarly be formed by an impression or cast (as described with reference to contact parts 180, 280). Using an impression or cast to form the contact parts 180, 280 and inner seal 401 may result in a more accurate reproduction of the shape of the subject, resulting in improved fit (and potentially improved sealing performance for the inner seal 401). The inner seal 401 may include any of the features described with reference to inner seal 400.

Specific example embodiments have been described in which there is a face part and neck part, which separate from each other (and engage with one another) on the frontal plane, other embodiments, according to the first or second aspect, are possible in which a first part and second part are provided that come together on the sagittal plane, and which are configured to conform with the subject in a similar way. Although example embodiments have been described in which there is a first part and second part that both conform to the subject, in some embodiments, according to the second aspect, only a single part may be configured to conform to the subject (which may be sufficient to accurately position the apparatus relative to the subject).

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination.

The examples provided in the detailed description are intended to provide examples of the invention, not to limit its scope, which should be determined with reference to the accompanying claims.

What is claimed is:

1.-32. (canceled)

33. A subject positioning apparatus, comprising:
a first part and a second part;
wherein the first part is configured to contact and conform with a region of the subject's head and/or neck, and to engage with and couple to a second part;
the first part is configured to hold the subject, when coupled to the second part, in a fixed position relative to the apparatus;
the first and second part each further comprising a portion of a seal flange, the seal flange of the first and second part together configured to clamp to further medical equipment so that a region of the subject's head above the seal flange is received in the further medical equipment.

34. The apparatus of claim 33, wherein the first part and second part are together configured to fix the apparatus with a positional accuracy, relative to the subject's neurocranium, of less than 5 mm.

35. The apparatus of claim 33, wherein the first part comprises a mating surface that is configured to engage with a corresponding mating surface of the second part.

36. The apparatus of claim 35, wherein the first and second part each comprise a mating flange that comprises the mating surface of the respective part, and the mating flange and/or mating surfaces of the first part and second part are substantially parallel to a frontal or sagittal plane of the subject.

37. The apparatus of claim 36, wherein the mating flange of the first part and second part comprises at least one slot for receiving a fastener that is arranged to clamp the mating surfaces of the first part and second part together.

38. The apparatus of claim 37, further comprising at least one fastener for clamping the mating surfaces of the first part and second part together, wherein the apparatus is configured for quick release of engagement between the first part and second part by loosening at least one fastener and sliding it out of the slot in the mating flange.

39. The apparatus of claim 33, wherein the first part and second part are customised to fit a specific subject.

40. The apparatus of claim 33, comprising a base portion having a base flange, the base flange configured to be received in a corresponding slot of a further medical apparatus, and/or comprising a mirror configured to allow the subject to see in an inferior or superior direction.

41. The apparatus of claim 33, wherein the seal flange is configured to engage with a sealing assembly that encircles the subject's head.

42. The apparatus of claim 41, wherein the seal flange is inclined at an angle of between 10 and 30 degrees to a transverse plane of the subject, and/or wherein the seal flange comprises one or more slots configured to receive a fastener for clamping the seal flange to a further medical apparatus.

43. The apparatus of claim 33, wherein the first part is a neck part and the first region of the subject's head comprises a posterior region of the subject's head, and the second part is a face part configured to contact and conform with a region of the subject's face.

44. The apparatus of claim 33, wherein the first part comprises a rigid portion consisting of a rigid material and a soft contact part comprising a softer material and/or the second part comprises a rigid portion consisting of a rigid material and a soft contact part comprising a softer material.

45. The apparatus of claim 44, wherein the contact part of the first part and/or the second part is formed from a cast or impression of the shape of the subject.

46. The apparatus of claim 33, further comprising a positioning cap, configured to temporarily engage with the first part or the second part, so as to better position the subject for the first and second parts to be coupled together.

47. The apparatus of claim 46, wherein the positioning cap comprises a head part that is configured to conform with the subject's upper cranium.

48. A method of locating a subject relative to medical equipment, comprising:
placing a first part in conformal contact with a first region of the subject's head and/or neck;
placing a second part in contact with a second portion of the head and/or neck;
fixing the first part to the second part;
wherein the first part and second part together form a subject positioning apparatus, and the method comprises slipping a seal assembly onto the subject's head adjacent to the subject positioning apparatus, and clamping the seal assembly between the subject positioning apparatus and the medical equipment.

49. The method of claim 48, wherein prior to fixing the first part to the second part, a positioning cap is coupled to the first part or second part, the positioning cap conforming with a top portion of the subject's cranium.

50. The method of claim 48, wherein, after fixing the first part to the second part, the positioning cap is detached from the first part or second part.

51. The method of claim 48, wherein the medical equipment comprises a cap comprising at least one ultrasonic transducer, and/or wherein the seal assembly is configured to form a seal with the subject's head and with the medical equipment, and the method comprises filling a space between the subject's head and the medical equipment with liquid and using the seal assembly to retain the liquid in contact with the medical equipment and the subject's head.

52. The method of claim 48 further comprising clamping the first part and second part to the medical equipment.
